(19) United States
(12) Patent Application Publication
(10) Pub. No.: US 2022/0208363 A1
(43) Pub. Date: Jun. 30, 2022

(54) MEDICAL SETTINGS PRESET SELECTION

(71) Applicant: JOHNSON & JOHNSON SURGICAL VISION, INC., Santa Ana, CA (US)

(72) Inventor: Vadim Gliner, Haifa (IL)

(21) Appl. No.: 17/520,968

(22) Filed: Nov. 8, 2021

Related U.S. Application Data
(60) Provisional application No. 63/130,536, filed on Dec. 24, 2020.

(51) Int. Cl. G16H 40/40; G16H 40/67

(52) U.S. Cl. CPC G16H 40/40; G16H 50/20; G16H 40/67

(57) ABSTRACT

In one embodiment, a therapeutic medical system includes treatment apparatuses disposed in respective locations interconnected via a network, each treatment apparatus including a medical tool configured to be inserted into a body part and operated according to a respective selected medical-tool-settings preset, a console configured to control the medical tool responsively to the respective selected medical-tool-settings preset, and a network interface to share data over the network, wherein the treatment apparatuses are configured to share, over the network, usage data of medical-tool-settings presets used by the treatment apparatuses, and a recommendation sub-system to receive the shared usage data of the medical-tool-settings presets, and find medical-tool-settings preset recommendations responsively to the shared usage data of the medical-tool-settings presets, wherein the console of a respective one of the treatment apparatuses is configured to render a respective one of the medical-tool-settings preset recommendations to the display of the respective treatment apparatus.

### RELATED APPLICATION INFORMATION

The present application claims benefit of U.S. Provisional Patent Application Ser. No. 63/130,536 of Vadim Gliner filed 24 Dec. 2020, the disclosure of which is hereby incorporated herein by reference.

### FIELD OF THE INVENTION

The present invention relates to medical systems, and in particular, but not exclusively to, medical tool settings.

### BACKGROUND

A cataract is a clouding and hardening of the eye's natural lens, a structure which is positioned behind the cornea, iris, and pupil. The lens is mostly made up of water and protein and as people age these proteins change and may begin to clump together obscuring portions of the lens. To correct this a physician may recommend phacoemulsification cataract surgery. Before the procedure, the surgeon numbs the area with anesthesia. Then a small incision is made in the sclera or clear cornea of the eye. Fluids are injected into this incision to support the surrounding structures. The anterior surface of the lens capsule is then removed to gain access to the cataract. The surgeon then uses a phacoemulsification probe, which has an ultrasonic handpiece with a titanium or steel needle. The tip of the needle vibrates at ultrasonic frequency to sculpt and emulsify the cataract while a pump aspirates lens particles and fluid from the eye through the tip. The pump is typically controlled with a microprocessor.

Any suitable pump may be used, for example, a peristaltic and/or a venturi type of pump. Aspirated fluids are replaced with irrigation of a balanced salt solution to maintain the anterior chamber of the eye. After removing the cataract with phacoemulsification, the softer outer lens cortex is removed with suction. An intraocular lens (IOL) is introduced into the empty lens capsule. Small struts called haptics hold the IOL in place. Once correctly installed the IOL restores the patient's vision.

### SUMMARY

There is provided in accordance with an embodiment of the present disclosure, a therapeutic medical system, including treatment apparatuses disposed in respective locations interconnected via a network, each of the treatment apparatuses including a medical tool configured to be inserted into a body part and operated a respective selected medical-tool-settings preset, a console configured to control the medical tool responsively to the respective selected medical-tool-settings preset, and a network interface configured to share data over the network, wherein the treatment apparatuses are configured to share, over the network, usage data of medical-tool-settings presets used by the treatment apparatuses, and a recommendation sub-system configured to receive the shared usage data of the medical-tool-settings presets, and find medical-tool-settings preset recommendations responsively to the shared usage data of the medical-tool-settings presets, wherein the console of a respective one of the treatment apparatuses is configured to render a respective one of the medical-tool-settings preset recommendations to the display of the respective treatment apparatus.

Further in accordance with an embodiment of the present disclosure the recommendation sub-system is configured to find respective ones of the medical-tool-settings preset recommendations for respective stages of a medical procedure.

Still further in accordance with an embodiment of the present disclosure the medical tool includes a phacoemulsification probe.

Additionally, in accordance with an embodiment of the present disclosure the medical-tool-settings-presets include any two or more of the following a respective vacuum setting, a respective aspiration rate setting, a respective pitch setting, a respective vibration mode setting, and a respective power setting.

Moreover, in accordance with an embodiment of the present disclosure the console is configured to render, to the display of the respective treatment apparatus, the respective medical-tool-settings preset recommendation with at least one different medical-tool-settings preset previously used by a user of the respective treatment apparatus.

Further in accordance with an embodiment of the present disclosure the recommendation sub-system is configured to find the medical-tool-settings preset recommendations responsively to a similarity between users of the treatment apparatuses and/or usage of the medical-tool-settings presets.

Still further in accordance with an embodiment of the present disclosure the recommendation sub-system is configured to maintain a data set including values indicating medical-tool-settings preset usage different combinations of users and the medical-tool-settings presets, infer medical-tool-settings preset usage values in the data set for different combinations of the users and the medical-tool-settings presets for which no medical-tool-settings preset usage currently exists, and find the medical-tool-settings preset recommendations responsively to ones of the inferred values.

Additionally, in accordance with an embodiment of the present disclosure the recommendation sub-system is configured to find the respective medical-tool-settings preset recommendation for a respective one of the users responsively to a highest one of the inferred values for the respective user.

Moreover, in accordance with an embodiment of the present disclosure the recommendation sub-system is configured to upon use of a respective one of the medical-tool-settings preset recommendations, increase a respective one of the inferred values, and upon use of another medical-tool-settings preset instead of a rendered one of the medical-tool-settings preset recommendations, reduce a respective one of the inferred values in the data set.

Further in accordance with an embodiment of the present disclosure the recommendation sub-system is configured to infer new medical-tool-settings preset usage values in the data set for different combinations of the users and the medical-tool-settings presets for which no medical-tool-settings preset usage currently exists and previously inferred values were not adjusted, and find new medical-tool-settings preset recommendations responsively to ones of the new inferred values.

Still further in accordance with an embodiment of the present disclosure the recommendation sub-system is configured to perform matrix factorization of a matrix including the data set including the values indicating the medical-tool-settings preset usage the different combinations of the users and the medical-tool-settings presets, and infer the medical-tool-settings preset usage values in the data set for different combinations of the users and the medical-tool-settings presets for which no medical-tool-settings preset usage currently exists responsively to the matrix factorization.

Additionally, in accordance with an embodiment of the present disclosure the recommendation sub-system is configured to input the data set into an artificial neural network (ANN), and iteratively adjust parameters of the ANN until an output of the ANN includes the input, the output including the inferred values.

Moreover, in accordance with an embodiment of the present disclosure the ANN includes an autoencoder.

There is also provided in accordance with another embodiment of the present disclosure a medical method, including receiving shared usage data of medical-tool-settings presets from treatment apparatuses disposed in respective locations interconnected via a network, finding medical-tool-settings preset recommendations responsively to the shared usage data of the medical-tool-settings presets, and rendering a respective one of the medical-tool-settings preset recommendations to a display of one of the respective treatment apparatuses.

Further in accordance with an embodiment of the present disclosure the finding includes finding respective ones of the medical-tool-settings preset recommendations for respective stages of a medical procedure.

Still further in accordance with an embodiment of the present disclosure the medical-tool-settings-preset includes any two or more of the following a respective vacuum setting, a respective aspiration rate setting, a respective pitch setting, a respective vibration mode setting, and a respective power setting.

Additionally, in accordance with an embodiment of the present disclosure the rendering includes rendering, to the display of the respective treatment apparatus, the respective medical-tool-settings preset recommendation with at least one different medical-tool-settings preset previously used by a user of the respective treatment apparatus.

Moreover, in accordance with an embodiment of the present disclosure the finding includes finding the medical-tool-settings preset recommendations responsively to a similarity between users of the treatment apparatuses and/or usage of the medical-tool-settings presets.

Further in accordance with an embodiment of the present disclosure, the method includes maintaining a data set including values indicating medical-tool-settings preset usage different combinations of users and the medical-tool-settings presets, and inferring medical-tool-settings preset usage values in the data set for different combinations of the users and the medical-tool-settings presets for which no medical-tool-settings preset usage currently exists, wherein the finding includes finding the medical-tool-settings preset recommendations responsively to ones of the inferred values.

Still further in accordance with an embodiment of the present disclosure the finding includes finding the respective medical-tool-settings preset recommendation for a respective one of the users responsively to a highest one of the inferred values for the respective user.

Additionally, in accordance with an embodiment of the present disclosure, the method includes upon use of a respective one of the medical-tool-settings preset recommendations, increasing a respective one of the inferred values, and upon use of another medical-tool-settings preset instead of a rendered one of the medical-tool-settings preset recommendations, reducing a respective one of the inferred values in the data set.

Moreover, in accordance with an embodiment of the present disclosure, the method includes inferring new medical-tool-settings preset usage values in the data set for different combinations of the users and the medical-tool-settings presets for which no medical-tool-settings preset usage currently exists and previously inferred values were not adjusted, and finding new medical-tool-settings preset recommendations responsively to ones of the new inferred values.

Further in accordance with an embodiment of the present disclosure, the method includes performing matrix factorization of a matrix including the data set including the values indicating the medical-tool-settings preset usage the different combinations of the users and the medical-tool-settings presets, wherein the inferring includes inferring the medical-tool-settings preset usage values in the data set for different combinations of the users and the medical-tool-settings presets for which no medical-tool-settings preset usage currently exists responsively to the matrix factorization.

Still further in accordance with an embodiment of the present disclosure, the method includes inputting the data set into an artificial neural network (ANN), and iteratively adjusting parameters of the ANN until an output of the ANN includes the input data set, the output including the inferred values.

Additionally, in accordance with an embodiment of the present disclosure the ANN includes an autoencoder.

### BRIEF DESCRIPTION OF THE DRAWINGS

The present invention will be understood from the following detailed description, taken in conjunction with the drawings in which:

FIG. 1 is a schematic pictorial illustration of an ophthalmic surgical system constructed and operative in accordance with an embodiment of the present invention;

FIG. 2 is a block diagram view of a therapeutic medical system constructed and operative in accordance with an embodiment of the present invention;

FIG. 3 is a flowchart including steps in a method of operation of a treatment apparatus in the system of FIG. 2;

FIG. 4 is a schematic view of setting presets rendered by one of the treatment apparatuses of FIG. 2;

FIG. 5 is a flowchart including steps in method of operation of a recommendation sub-system in the system of FIG. 2;

FIG. 6 is a schematic illustration of matrix factorization for use in the method of FIG. 5;

FIGS. 7 and 8 are schematic illustrations showing updating entries in the matrix of FIG. 6; and

FIG. 9 is a schematic view of an artificial neural network for use in a recommendation sub-system in the system of FIG. 2.

### DESCRIPTION OF EXAMPLE EMBODIMENTS

### Overview

A therapeutic medical system (for example, a phacoemulsification system or an ablation system) may present a physician with various medical-tool-settings presets for selection. For example, in a phacoemulsification system, each preset may include a power setting, a vacuum setting, an aspiration rate setting, pitch, vibration mode etc. It can be very confusing for a physician to select among the given presets. Therefore, physicians generally use the same presets that they are familiar with and ignore the rest. However, many useful presets, which may enhance the medical procedure (e.g., phacoemulsification or ablation procedure), may be overlooked by the physician due to the large selection of available presets.

Embodiments of the present invention solve the above problems by recommending one or more different medical-tool-settings presets not used by the physician previously along with one or more presets (e.g., most popular presets) recently used by the physician. The recommendation of presets may be performed using any recommendation method based on similarity between users and/or presets using any suitable content and/or collaborative based filtering method. For this purpose, local medical treatment apparatuses (e.g., phacoemulsification or ablation apparatuses) are connected to a remote recommendation sub-system (e.g., recommendation engine) to which the local medical treatment apparatuses send usage data of the medical-tool-setting presets. In some embodiments, the recommendation sub-system may be distributed among some, or all, of the local medical treatment apparatuses as described in disclosed embodiments.

The recommendation sub-system provides preset recommendations to the local medical treatment apparatuses. In some embodiments, if a recommended preset is selected for use by the physician, the preset will receive a higher “score” in the system, and if it is not selected, the preset will receive a lower “score” in the system. The recommendation sub-system then uses the latest score data to update its recommendations to this physician and other users.

In some embodiments, recommendations are based on matrix factorization of a matrix of users versus presets with a value being assigned to each combination of user/preset in the matrix according to the usage of each preset by each user. However, if a user has not used a preset, the value for that preset will be missing and the matrix is incomplete. Therefore, matrix factorization may be performed using an iterative process which guesses the matrix factors of the incomplete matrix based on the known values of the matrix. The matrix factors may then be used to determine the missing values by multiplying together the found matrix factors. The “missing” values (also referred to as “inferred” values) may then be used to recommend presets, for example, based on the highest scoring preset(s) of those “missing” scores. For example, if there are two inferred values for user X, with one of the inferred values being equal to 3 for preset A and one of the inferred values being equal to 5 for preset B, then preset B will be selected by the recommendation sub-system and sent to user X as a recommended preset. If a recommended preset (e.g., preset B) is selected by the physician (e.g., User X), the preset (e.g., preset B) will receive a higher score (e.g., 6) in the matrix, and if it is not selected, the preset (e.g., preset B) will receive a lower score (e.g., 1) in the matrix. The recommendation sub-system then uses the latest usage data from all users to update the matrix and provide new recommendations to all users based on the new matrix.

In some embodiments, instead of using matrix factorization described above, an artificial neural network (ANN) (e.g., autoencoder) may be used to find the “inferred” values from the known preset usage values. The known preset usage values are input into the ANN with the input data being ordered according different combinations of users and presets with gaps in the appropriate places for combinations of users and presets without corresponding usage data. The parameters of the ANN are iteratively adjusted until an output of the ANN includes the known preset usage values. At that point, the output of the ANN then also includes the inferred values (in the place of the gaps), which may then be used to provide preset recommendations.

### System Description

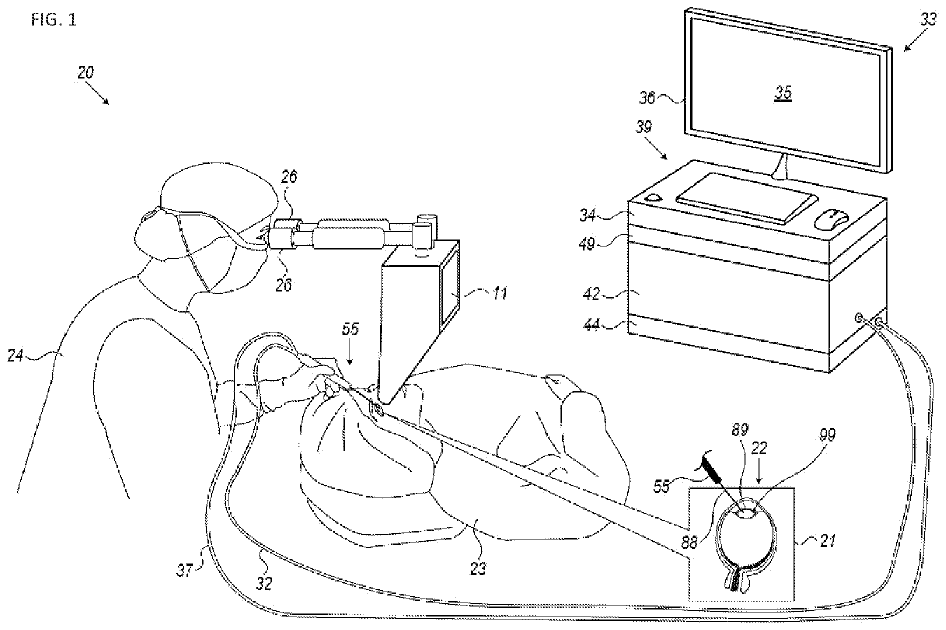

FIG. 1 is a schematic pictorial illustration of an ophthalmic surgical system 20, in accordance with an embodiment of the present invention. System 20 is configured to carry out various types of ophthalmic procedures, such as cataract surgery.

In some embodiments, system 20 comprises a medical instrument, in the present example a phacoemulsification handpiece, also referred to herein as a tool 55, used by a surgeon 24 to carry out the cataract surgery. In other embodiments, system 20 may comprise other surgical tools, such as but not limited to an irrigation and aspiration (I/A) handpiece, a diathermy handpiece, a vitrectomy handpiece, and similar instruments.

Reference is now made to an inset 21 showing a sectional view of the surgical procedure carried out in an eye 22 of a patient 23. In some embodiments, surgeon 24 applies tool 55 for treating eye 22, and in the present example, surgeon 24 inserts a needle 88 of tool 55 into eye 22. In the example of inset 21, during a cataract surgical procedure, surgeon 24 inserts needle 88 into a capsular bag 89 so as to emulsify a lens 99 of eye 22.

Reference is now made back to the general view of FIG. 1. In some embodiments, system 20 comprises a console 33, which comprises a processor 34, a memory 49, a generator 44 and a cartridge 42. In some embodiments, cartridge 42 comprises pumping sub-systems (not shown) configured to apply, via multiple tubes 32, irrigation fluids (not shown) into eye 22 and to draw eye fluids away from eye 22 into cartridge 42. In the context of the present invention, the term “eye fluid” refers to any mixture of natural eye fluid, irrigation fluid and lens material. Note that tubes 32 may comprise an irrigation tube for supplying the irrigation fluid into eye 22, and a separate aspiration tube for drawing the eye fluids away from eye 22.

In some embodiments, generator 44 is electrically connected to tool 55, via a plurality of wires referred to herein as an electrical cable 37. Generator 44 is configured to generate one or more voltage periodic (e.g., sinusoidal) signals, also referred to herein as periodic signals, having one or more frequencies, respectively. Generator 44 is further configured to generate a plurality of driving signals, so as to vibrate needle 88 of tool 55 in accordance with a predefined pattern, so as to emulsify lens 99 of eye 22.

In some embodiments, processor 34 typically comprises a general-purpose computer, with suitable front end and interface circuits for controlling generator 44, cartridge 42 and other components of system 20.

In practice, some or all of the functions of the processor 34 may be combined in a single physical component or, alternatively, implemented using multiple physical components. These physical components may comprise hard-wired or programmable devices, or a combination of the two. In some embodiments, at least some of the functions of the processor 34 may be carried out by a programmable processor under the control of suitable software. This software may be downloaded to a device in electronic form, over a network, for example. Alternatively, or additionally, the software may be stored in tangible, non-transitory computer-readable storage media, such as optical, magnetic, or electronic memory.

In some embodiments, system 20 comprises an ophthalmic surgical microscope 11, such as ZEISS OPMI LUMERA series or ZEISS ARTEVO series supplied by Carl Zeiss Meditec AG (Oberkochen, Germany), or any other suitable type of ophthalmic surgical microscope provided by other suppliers. Ophthalmic surgical microscope 11 is configured to produce stereoscopic optical images and two-dimensional (2D) optical images of eye 22. During the cataract surgery, surgeon 24 typically looks though eyepieces 26 of ophthalmic surgical microscope 11 for viewing eye 22.

In some embodiments, console 33 comprises a display 36 and input devices 39, which may be used by surgeon 24 for controlling tool 55 and other components of system 20. Moreover, processor 34 is configured to display on display 36, an image 35 received from any suitable medical imaging system for assisting surgeon to carry out the cataract surgery.

This particular configuration of system 20 is shown by way of example, in order to illustrate certain problems that are addressed by embodiments of the present invention and to demonstrate the application of these embodiments in enhancing the performance of such a system. Embodiments of the present invention, however, are by no means limited to this specific sort of example system, and the principles described herein may similarly be applied to other sorts of ophthalmic and other minimally invasive and surgical systems.

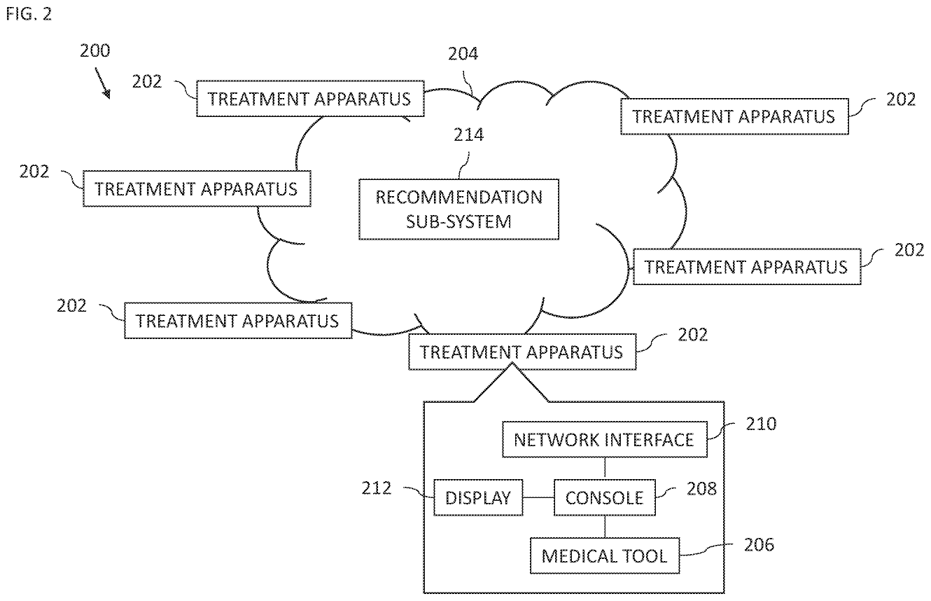

Reference is now made to FIG. 2, which is a block diagram view of a therapeutic medical system 200 constructed and operative in accordance with an embodiment of the present invention. The therapeutic medical system 200 includes treatment apparatuses 202 disposed in respective locations interconnected via a network 204. Each of the treatment apparatuses 202 includes a medical tool 206, a console 208, a network interface 210, and a display 212.

The medical tool 206 may include any suitable medical tool, for example, a phacoemulsification probe (e.g., the tool 55 of FIG. 1) or a catheter for performing tissue ablation or a diathermy tool to perform coagulation. The medical tool 206 is configured to be inserted into a body part (e.g., the capsular bag 89 of FIG. 1 or a chamber of the heart) and operated according to a respective selected medical-tool-settings preset.

The console 208 is configured to control the medical tool 206 responsively to the respective selected medical-tool-settings preset. The console 208 may include various elements to control the medical tool 206 such as a processor (e.g., processor 34), memory (e.g., memory 49), a signal generator (e.g., generator 44), pumps (e.g., to perform aspiration and irrigation). The medical-tool-settings-presets may include any two or more of the following: a respective vacuum setting; a respective aspiration rate setting; a respective pitch setting (e.g., to control the longitudinal extent of the needle 88); a respective vibration mode setting (e.g., of the needle 88, such as traversal, longitudinal); a respective power setting (e.g., needle power setting, or ablation power setting); ablation duration, and other phacoemulsification or ablation settings.

The treatment apparatuses 202 are configured to share, over the network 204, usage data of medical-tool-settings presets used by the treatment apparatuses 202. In some embodiments, the network interface 210 of each treatment apparatus 202 is configured to share data of medical-tool-settings preset usage over the network 204 to a recommendation sub-system 214 of the therapeutic medical system 200. The usage data generally indicates how many times each user has used each of the medical-tool-settings presets.

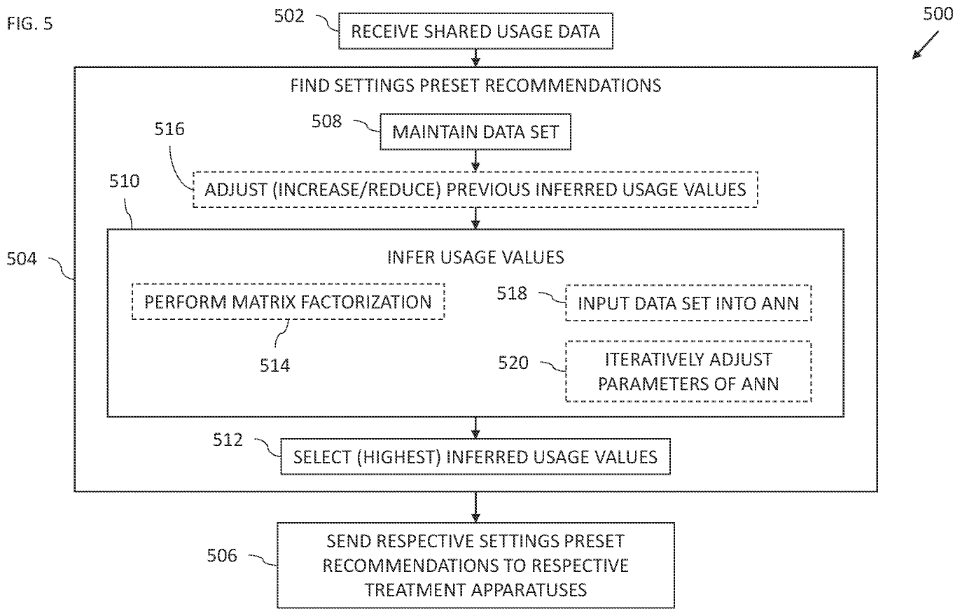

The recommendation sub-system 214 is configured to receive the shared usage data of the medical-tool-settings presets. The recommendation sub-system 214 is configured to find medical-tool-settings preset recommendations responsively to the shared usage data of the medical-tool-settings presets, as described in more detail with reference to FIGS. 5-9. The recommendation sub-system 214 is configured to send the medical-tool-settings preset recommendations to the respective treatment apparatuses 202.

In some embodiments, the recommendation sub-system 214 is configured as a central processing server which collects and processes the preset usage data received from the treatment apparatuses 202, finds preset recommendations, and sends the preset recommendations to the different treatment apparatuses 202.

In some embodiments, the recommendation sub-system 214 is configured as a central server which collects the preset usage data from the treatment apparatuses 202 and sends the collected preset usage data to the treatment apparatuses 202 so that each of the treatment apparatuses 202 may find local recommendations based on the received preset usage data.

In other embodiments, the recommendation sub-system 214 is distributed among the treatment apparatuses 202 without a central processing server. In these other embodiments, the preset usage data is shared among the treatment apparatuses 202 so that each of the treatment apparatuses 202 may find local recommendations based on the received preset usage data.

The console 208 of a respective treatment apparatus 202 is configured to render a respective medical-tool-settings preset recommendation (i.e., the recommendation found for that treatment apparatus 202) to the display 212 of the respective treatment apparatus 202 optionally with one or more different medical-tool-settings presets previously used by a user of the respective treatment apparatus 202. In a similar fashion, other treatment apparatuses 202 render their preset recommendation(s) to their respective displays 212.

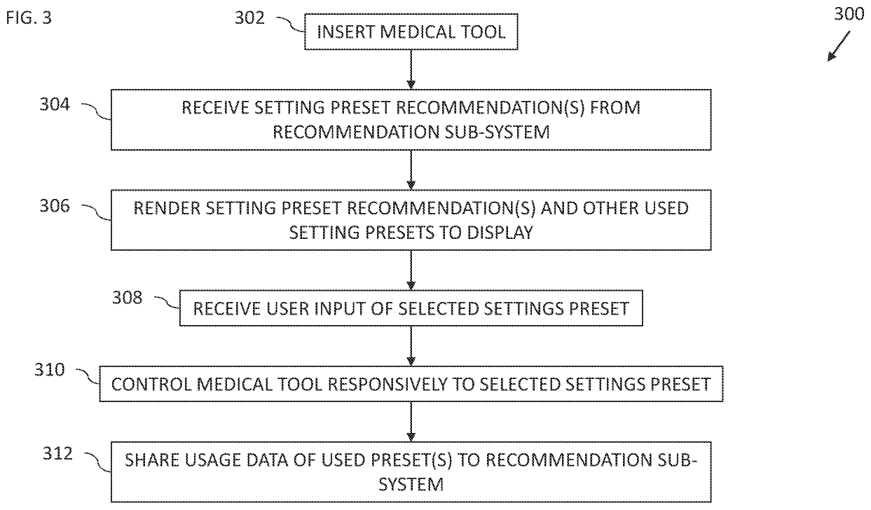

Reference is now made to FIG. 3, which is a flowchart 300 including steps in a method of operation of one of the treatment apparatuses 202 in the system 200 of FIG. 2. Reference is also made to FIG. 2. The physician inserts (block 302) the medical tool 206 into the body part (e.g., capsular bag 89 or heart chamber) of the patient. The console 208 receives (block 304) a medical-tool-settings preset recommendation from the recommendation sub-system 214.

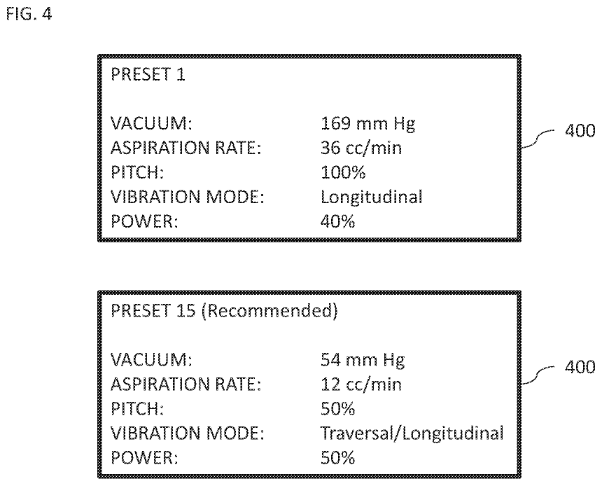

Reference is now made to FIG. 4, which is a schematic view of settings presets 400 rendered by one of the treatment apparatuses of FIG. 2. Reference is also made to FIG. 3. The console 208 renders (block 306) the recommended medical-tool-settings preset (optionally with one or more different medical-tool-settings presets previously used by a user of the respective treatment apparatus 202) to the display 212. The console 208 receives (block 308) a user input of the selected settings preset. The console 208 controls (block 310) the medical tool 206 responsively to the settings of the selected medical-tool-settings preset. The console 208 shares (block 312) usage data of the used preset(s) to the recommendation sub-system 214 (or to the other treatment apparatuses 202).

In practice, some or all of the functions of the console 208 may be combined in a single physical component or, alternatively, implemented using multiple physical components. These physical components may comprise hard-wired or programmable devices, or a combination of the two. In some embodiments, at least some of the functions of the console 208 may be carried out by a programmable processor under the control of suitable software. This software may be downloaded to a device in electronic form, over a network, for example. Alternatively, or additionally, the software may be stored in tangible, non-transitory computer-readable storage media, such as optical, magnetic, or electronic memory.

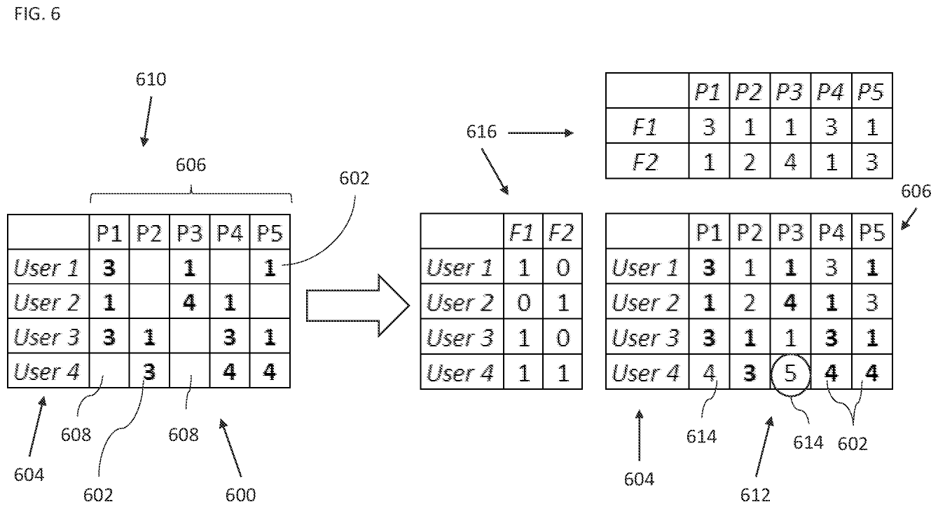

Reference is now made to FIG. 5, which is a flowchart 500 including steps in method of operation of the recommendation sub-system 214 in the therapeutic medical system 200 of FIG. 2. The recommendation sub-system 214 is configured to receive (block 502) shared usage data of the medical-tool-settings presets by the users of the treatment apparatuses 202. The recommendation sub-system 214 is configured to find (block 504) medical-tool-settings preset recommendations responsively to the received shared usage data of the medical-tool-settings presets and typically a similarity between users of the treatment apparatuses and/or usage of the medical-tool-settings presets. The recommendation sub-system 214 may find recommendations from the preset usage data responsively to any suitable recommendation algorithm, for example, based on collaborative and/or content-based filtering. For example, if two users have similar user profiles, a preset of one of the users may be suggested to the other user. By way of another example, if two users use many of the same presets, a preset used by one of the users, but not the other, may be recommended to the other user. The step of block 504 is described in more detail with reference to FIGS. 6-9. The recommendation sub-system 214 is configured to send (block 506) respective found medical-tool-settings preset recommendations to respective treatment apparatuses 202. In other words, the preset recommendation(s) for the user of one of the treatment apparatuses 202, is sent to that treatment apparatus 202, and so on.

Reference is now made to FIG. 6, which is a schematic illustration of matrix factorization for use in the method of FIG. 5. Reference is also made to FIG. 5.

The recommendation sub-system 214 is configured to maintain (block 508) a data set 600 comprising values 602 (only some labelled for the sake of simplicity) indicating medical-tool-settings preset usage according to different combinations of users and the medical-tool-settings presets responsively to the received shared medical-tool-settings preset usage data. The data set 600 is shown in the form of a matrix 610 of users 604 (e.g., users 1, user 2, and so on) versus presets 606 (e.g., preset P1, preset P2, and so on). The data set 600 may be shown in any suitable form, for example, a string of values. By way of example, user 4 has used preset P2 three times. Some of the presets 606 have not been used by the users 604, signified by missing entries 608 (only some shown for the sake of simplicity). For example, presets P1 and P3 have not been used by user 4. The data set 600 is updated by the recommendation sub-system 214 as new preset usage data is received from the treatment apparatuses 202.

The recommendation sub-system 214 is configured to infer (block 510) medical-tool-settings preset usage values 614 in the data set for different combinations of the users 604 and the medical-tool-settings presets 606 for which no medical-tool-settings preset usage currently exists (e.g., for missing entries 608). For example, the recommendation sub-system 214 infers the usage value for presets P1 and P3 for user 4. The inferred usage values 614 provide indications of how many times the relevant presets 606 would likely be used by the users 604 and may therefore be used to provide preset recommendations to the users 604. FIG. 6 shows a second matrix 612 which includes the known usage values 602 (shown in bold, and only some labeled for the sake of simplicity), and inferred usage values 614 (not shown in bold, and only some labeled for the sake of simplicity). For example, the inferred usage value 614 for preset P1 of user 4 is equal to 4, and the inferred usage value 614 for preset P3 of user 4 is equal to 5 (circled). Therefore, of the presets 606 not previously used by user 4, preset P3 receives a higher inferred usage value 614 equal to 5. The recommendation sub-system 214 is configured to find medical-tool-settings preset recommendations responsively to at least some of the inferred values 614. In some embodiments, the recommendation sub-system 214 is configured to find the respective medical-tool-settings preset recommendation for a respective user (e.g., user 4) responsively to selecting (block 512) the highest inferred value 614 for that user. For example, the highest inferred usage value 614 for user 4 is for preset P3 and is equal to 5 (circled in FIG. 6).

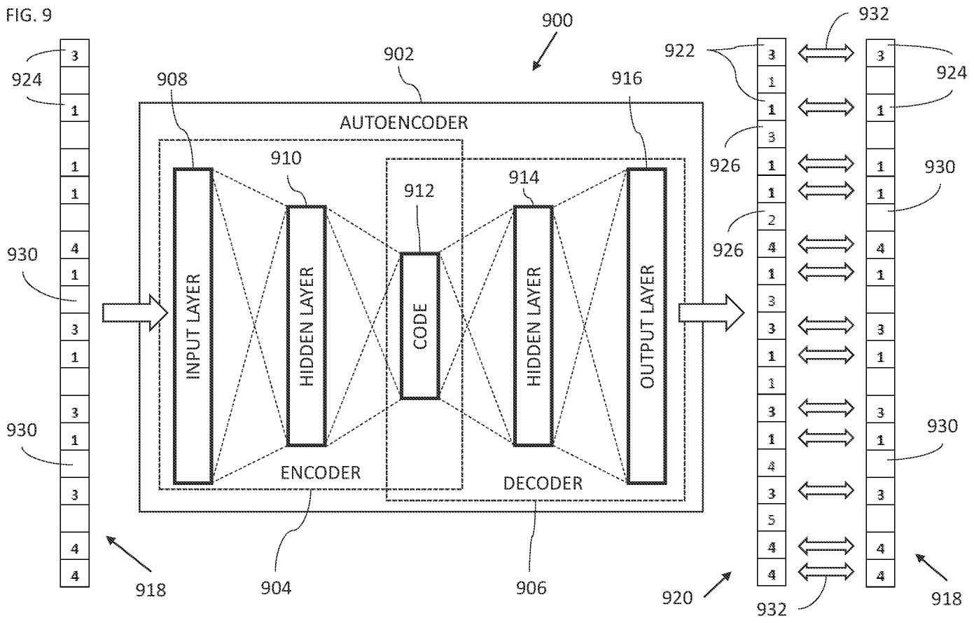

The recommendation sub-system 214 may infer the usage values 614 using any suitable method, for example, using matrix factorization described in more detail below, or using an artificial neural network (ANN) such as an autoencoder described in more detail with reference to FIG. 9.

The recommendation sub-system 214 is configured to perform matrix factorization (block 514) of the incomplete matrix 610 including the data set 600 (comprising the values 602 indicating the medical-tool-settings preset usage according to the different combinations of the users 604 and the medical-tool-settings presets 606). The matrix factorization may include an iterative process in which factors 616 of the matrix 610 are guessed and iteratively adjusted until the factors 616 multiply to give a matrix 612 which includes the data set 600 (without consideration of the values of the missing entries 608). Once the factors 616 of the matrix 610 including the data set 600 are found, multiplying the factors 616 provides the matrix 612 with the inferred usage values 614 instead (i.e., in place) of the missing entries 608. Therefore, the recommendation sub-system 214 is configured to infer the medical-tool-settings preset usage values 614 in the data set 600 for different combinations of the users 604 and the medical-tool-settings presets 606 for which no medical-tool-settings preset usage currently exists responsively to the matrix factorization.

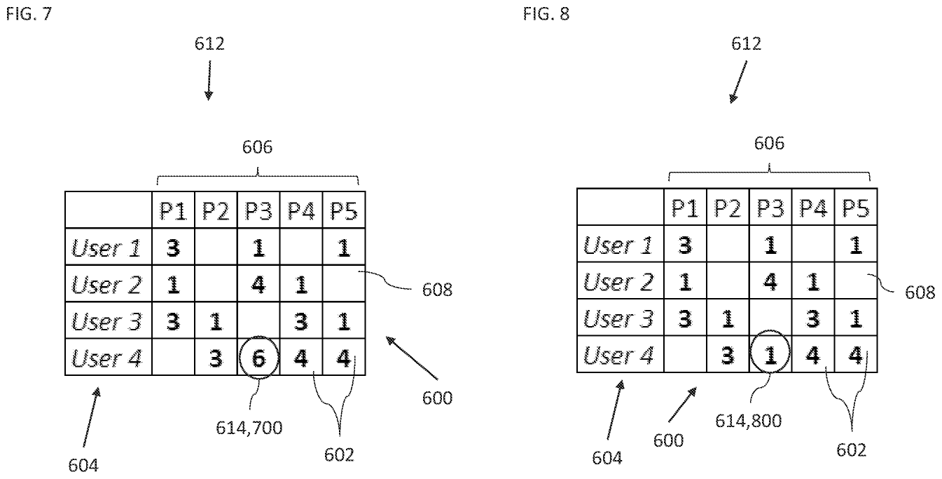

Reference is now made to FIGS. 7 and 8, which are schematic illustrations showing updating entries in the matrix 612 of FIG. 6. Reference is also made to FIG. 5. As previously explained, medical-tool-settings presets 606 are recommended to the treatment apparatuses 202. One or more respective recommended presets 606 are displayed by each respective treatment apparatus 202 with one or more other presets 606 previously used by the user 604 of the respective treatment apparatus 202. If a recommended preset 606 is selected and used by the user 604, then the usage value for that preset 606 is increased (with respect to the inferred usage value 614 for that preset and user) in the matrix 612. If the recommended preset is not selected (e.g., one of the other presets is selected for use), then the usage value for that preset 606 is reduced (with respect to the inferred usage value 614 for that preset and user) in the matrix 612. For example, if preset P3 recommended to user 4 is selected for use by user 4 then the inferred usage value 614 for preset P3 and user 4 equal to 5 (circled in FIG. 6) is increased, for example, to 6 (circle 700), as shown in FIG. 7, whereas if preset P3 recommended to user 4 is not selected for use by user 4, then the inferred usage value 614 for preset P3 and user 4 equal to 5 (circled in FIG. 6) is reduced, for example, to 1 (circle 800), as shown in FIG. 8. The matrix 612 is shown without the other inferred usage values 614 from FIG. 6.

Therefore, the recommendation sub-system 214 is configured to adjust (block 516 of FIG. 5) previous inferred usage values 614 based on user selections of recommendations of corresponding presets 606. The recommendation sub-system 214 is configured upon use of a respective medical-tool-settings preset recommendation (respective of a user 604 and preset 606 combination), to increase the respective inferred value 614 (respective of that user 604 and preset 606 combination). The recommendation sub-system 214 is configured upon use of another medical-tool-settings preset instead of the rendered medical-tool-settings preset recommendation (respective of a user and preset combination), to reduce a respective inferred value 614 in the data set 600 (for that preset 606 and user 604 combination). Once adjusted, the inferred usage values 614 are considered like the actual usage values 602 and are retained in the matrix 612 while other unadjusted inferred usage values 614 are removed from the matrix 612 prior to inferring new values from the incomplete matrix 612.

After the inferred usage values 614 are updated (responsively to selection and non-selection of preset recommendations) and optionally new preset usage data is received from the network 204, the previous unadjusted inferred usage values 614 are removed from the data set 600 leaving updated usage data (e.g., values 602), adjusted inferred usage values 614 (responsively to selection and non-selection of preset recommendations), and missing entries 608.

The recommendation sub-system 214 is configured to: infer (e.g., by repeating the step of block 510) new medical-tool-settings preset usage values 614 in the data set 600 for different combinations of the users 604 and the medical-tool-settings presets 606 for which no medical-tool-settings preset usage currently exists and previously inferred values 614 were not adjusted; and find (e.g., by repeating the step of block 512) new medical-tool-settings preset recommendations responsively to at least some of the new inferred values.

In some embodiments, the recommendation sub-system 214 is configured to find respective medical-tool-settings preset recommendations for respective stages of a medical procedure (e.g., grooving and chopping during cataract surgery) even with the same medical tool. It should be noted that the preset recommendations found for the different stages may include one or more common presets that are recommended in more than one stage of the medical procedure (for the same user or different users). Therefore, the recommendation sub-system 214 may maintain different respective preset usage datasets for different respective stages of the medical procedure and provide preset recommendations for the respective stages of the medical procedure responsively to the respective datasets.

Reference is now made to FIG. 9, which is a schematic view of an artificial neural network 900 for use in the recommendation sub-system 214 in the system 200 of FIG. 2. Reference is also made to FIG. 5.

A neural network is a network or circuit of neurons, or in a modern sense, an artificial neural network, composed of artificial neurons or nodes. The connections of the biological neuron are modeled as weights. A positive weight reflects an excitatory connection, while negative values mean inhibitory connections. Inputs are modified by a weigh t and summed using a linear combination. An activation function may control the amplitude of the output.

These artificial networks may be used for predictive modeling, adaptive control and applications and can be trained via a dataset. Self-learning resulting from experience can occur within networks, which can derive conclusions from a complex and seemingly unrelated set of information.

For completeness, a biological neural network is composed of a group or groups of chemically connected or functionally associated neurons. A single neuron may be connected to many other neurons and the total number of neurons and connections in a network may be extensive. Connections, called synapses, are usually formed from axons to dendrites, though dendrodendritic synapses and other connections are possible. Apart from the electrical signaling, there are other forms of signaling that arise from neurotransmitter diffusion.

Artificial intelligence, cognitive modeling, and neural networks are information processing paradigms inspired by the way biological neural systems process data. Artificial intelligence and cognitive modeling try to simulate some properties of biological neural networks. In the artificial intelligence field, artificial neural networks have been applied successfully to speech recognition, image analysis and adaptive control, in order to construct software agents (in computer and video games) or autonomous robots.

A neural network (NN), in the case of artificial neurons called artificial neural network (ANN) or simulated neural network (SNN), is an interconnected group of natural or artificial neurons that uses a mathematical or computational model for information processing based on a connectionistic approach to computation. In most cases an ANN is an adaptive system that changes its structure based on external or internal information that flows through the network. In more practical terms, neural networks are non-linear statistical data modeling or decision-making tools. They can be used to model complex relationships between inputs and outputs or to find patterns in data.

In some embodiments, as shown in FIG. 9, the artificial neural network 900 may include an autoencoder 902 including an encoder 904 and a decoder 906. In other embodiments, the artificial neural network 900 may comprise any suitable ANN. The artificial neural network 900 may be implemented in software and/or hardware.

The encoder 904 includes an input layer 908 into which an input is received. The encoder 904 then includes one or more hidden layers 910 which progressively compress the input to a code 912. The decoder 906 includes one or more hidden layers 914 which progressively decompress the code 912 up to an output layer 916 from which the output of the autoencoder 902 is provided. The autoencoder 902 includes weights between the layers of the autoencoder 902. The autoencoder 902 manipulates the data received at the input layer 908 according to the values of the various weights between the layers of the autoencoder 902. The weights of the autoencoder 902 are updated during use of the autoencoder 902 as described in more detail below.

The number of layers in the autoencoder 902 and the width of the layers may be configurable. As the number of layers and width of the layers increases so does the accuracy to which the autoencoder 902 can manipulate data according to the task at hand. By way of example, the input layer 908 may include 400 neurons (e.g., to compress a batch of 400 samples). The encoder 904 may include five layers which compress by a factor of two (e.g., 400, 200, 100, 50, 25). The decoder 906 may include five layers which decompress by a factor of 2 (e.g., 25, 50, 100, 200, 400).

The recommendation sub-system 214 is configured to: input (block 518 of FIG. 5) a data set 918 (which corresponds to the data set 600) into the artificial neural network 900; and iteratively adjust (block 520 of FIG. 5) parameters of the ANN 900 until an output 920 of the ANN 900 includes the input data set 918. The input data set 918 includes values 924 (only some labeled for the sake of simplicity) and gaps 930 (only some labeled for the sake of simplicity) between the values 924, the gaps 930 corresponding with missing values in the data set 918. The data in the input data set 918 is ordered according to different combinations of users and presets. In the example of FIG. 9, the input data set 918 first includes the usage values of user 1, followed by the usage values of user 2 and follow on. The parameters of the artificial neural network 900 are adjusted until values 922 (in bold) (only some labeled for the sake of simplicity) included in the output 920 are the same as the corresponding values 924 in the input data set 918 taking into account the order of the data and the relevant gaps 930. The comparison of only some of the values in output 920 to corresponding values in the data set 918 is indicated using arrows 932 (only some labeled for the sake of simplicity). The other values 926 in the output 920 (corresponding to the gaps 930 in the input data set 918) (only some labeled for the sake of simplicity) are not compared to the input.

The comparison is generally performed using a suitable loss function, which computes the overall difference between all the relevant outputs (e.g., the values 922) of the artificial neural network 900 and all the desired outputs (e.g., all the corresponding values 924 of the input data set 918). The recommendation sub-system 214 is configured to amend the parameters of the artificial neural network 900 using any suitable optimization algorithm, for example, a gradient descent algorithm such as Adam Optimization.

Once the parameters of the artificial neural network 900 have been adjusted so that the values 922 in the output 920 are equal to the corresponding values 924 in the input data set 918, the other values 926 in the output 920 then correspond with inferred usage values (which correspond to the inferred usage values 614) corresponding with the gaps 930.

In practice, some or all of these functions may be combined in a single physical component or, alternatively, implemented using multiple physical components. These physical components may comprise hard-wired or programmable devices, or a combination of the two. In some embodiments, at least some of the functions of the processing circuitry may be carried out by a programmable processor under the control of suitable software. This software may be downloaded to a device in electronic form, over a network, for example. Alternatively, or additionally, the software may be stored in tangible, non-transitory computer-readable storage media, such as optical, magnetic, or electronic memory.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 72% to 108%.

Various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination.

The embodiments described above are cited by way of example, and the present invention is not limited by what has been particularly shown and described hereinabove. Rather the scope of the invention includes both combinations and sub-combinations of the various features described hereinabove, as well as variations and modifications thereof which would occur to persons skilled in the art upon reading the foregoing description and which are not disclosed in the prior art.

What is claimed is:

1. A therapeutic medical system, comprising:
treatment apparatuses disposed in respective locations interconnected via a network, each of the treatment apparatuses comprising:
a medical tool configured to be inserted into a body part and operated according to a respective selected medical-tool-settings preset;
a console configured to control the medical tool responsively to the respective selected medical-tool-settings preset; and
a network interface configured to share data over the network, wherein the treatment apparatuses are configured to share, over the network, usage data of medical-tool-settings presets used by the treatment apparatuses; and
a recommendation sub-system configured to receive the shared usage data of the medical-tool-settings presets; and find medical-tool-settings preset recommendations responsively to the shared usage data of the medical-tool-settings presets, wherein the console of a respective one of the treatment apparatuses is configured to render a respective one of the medical-tool-settings preset recommendations to the display of the respective treatment apparatus.

2. The system according to claim 1, wherein the recommendation sub-system is configured to find respective ones of the medical-tool-settings preset recommendations for respective stages of a medical procedure.

3. The system according to claim 1, wherein the medical tool comprises a phacoemulsification probe.

4. The system according to claim 3, wherein the medical-tool-settings-presets include any two or more of the following: a respective vacuum setting; a respective aspiration rate setting; a respective pitch setting; a respective vibration mode setting; and a respective power setting.

5. The system according to claim 1, wherein the console is configured to render, to the display of the respective treatment apparatus, the respective medical-tool-settings preset recommendation with at least one different medical-tool-settings preset previously used by a user of the respective treatment apparatus.

6. The system according to claim 1, wherein the recommendation sub-system is configured to find the medical-tool-settings preset recommendations responsively to a similarity between users of the treatment apparatuses and/or usage of the medical-tool-settings presets.

7. The system according to claim 6, wherein the recommendation sub-system is configured to:
maintain a data set comprising values indicating medical-tool-settings preset usage according to different combinations of users and the medical-tool-settings presets;
infer medical-tool-settings preset usage values in the data set for different combinations of the users and the medical-tool-settings presets for which no medical-tool-settings preset usage currently exists; and
find the medical-tool-settings preset recommendations responsively to ones of the inferred values.

8. The system according to claim 7, wherein the recommendation sub-system is configured to find the respective medical-tool-settings preset recommendation for a respective one of the users responsively to a highest one of the inferred values for the respective user.

9. The system according to claim 7, wherein the recommendation sub-system is configured to:
upon use of a respective one of the medical-tool-settings preset recommendations, increase a respective one of the inferred values; and
upon use of another medical-tool-settings preset instead of a rendered one of the medical-tool-settings preset recommendations, reduce a respective one of the inferred values in the data set.

10. The system according to claim 9, wherein the recommendation sub-system is configured to:
infer new medical-tool-settings preset usage values in the data set for different combinations of the users and the medical-tool-settings presets for which no medical-tool-settings preset usage currently exists and previously inferred values were not adjusted; and
find new medical-tool-settings preset recommendations responsively to ones of the new inferred values.

11. The system according to claim 7, wherein the recommendation sub-system is configured to:
perform matrix factorization of a matrix including the data set comprising the values indicating the medical-tool-settings preset usage according to the different combinations of the users and the medical-tool-settings presets; and
infer the medical-tool-settings preset usage values in the data set for different combinations of the users and the medical-tool-settings presets for which no medical-tool-settings preset usage currently exists responsively to the matrix factorization.

12. The system according to claim 7, wherein the recommendation sub-system is configured to:
input the data set into an artificial neural network (ANN); and
iteratively adjust parameters of the ANN until an output of the ANN includes the input, the output including the inferred values.

13. The system according to claim 12, wherein the ANN includes an autoencoder.

14. A medical method, comprising:
receiving shared usage data of medical-tool-settings presets from treatment apparatuses disposed in respective locations interconnected via a network;
finding medical-tool-settings preset recommendations responsively to the shared usage data of the medical-tool-settings presets; and
rendering a respective one of the medical-tool-settings preset recommendations to a display of one of the respective treatment apparatuses.

15. The method according to claim 14, wherein the finding includes finding respective ones of the medical-tool-settings preset recommendations for respective stages of a medical procedure.

16. The method according to claim 14, wherein the medical-tool-settings-preset includes any two or more of the following: a respective vacuum setting; a respective aspiration rate setting; a respective pitch setting; a respective vibration mode setting; and a respective power setting.

17. The method according to claim 14, wherein the rendering includes rendering, to the display of the respective treatment apparatus, the respective medical-tool-settings preset recommendation with at least one different medical-tool-settings preset previously used by a user of the respective treatment apparatus.

18. The method according to claim 14, wherein the finding includes finding the medical-tool-settings preset recommendations responsively to a similarity between users of the treatment apparatuses and/or usage of the medical-tool-settings presets.

19. The method according to claim 18, further comprising:
maintaining a data set comprising values indicating medical-tool-settings preset usage according to different combinations of users and the medical-tool-settings presets; and
inferring medical-tool-settings preset usage values in the data set for different combinations of the users and the medical-tool-settings presets for which no medical-tool-settings preset usage currently exists, wherein the finding includes finding the medical-tool-settings preset recommendations responsively to ones of the inferred values.

20. The method according to claim 19, wherein the finding includes finding the respective medical-tool-settings preset recommendation for a respective one of the users responsively to a highest one of the inferred values for the respective user.

21. The method according to claim 19, further comprising:
upon use of a respective one of the medical-tool-settings preset recommendations, increasing a respective one of the inferred values; and
upon use of another medical-tool-settings preset instead of a rendered one of the medical-tool-settings preset recommendations, reducing a respective one of the inferred values in the data set.

22. The method according to claim 21, further comprising:
inferring new medical-tool-settings preset usage values in the data set for different combinations of the users and the medical-tool-settings presets for which no medical-tool-settings preset usage currently exists and previously inferred values were not adjusted; and
finding new medical-tool-settings preset recommendations responsively to ones of the new inferred values.

23. The method according to claim 19, further comprising performing matrix factorization of a matrix including the data set comprising the values indicating the medical-tool-settings preset usage according to the different combinations of the users and the medical-tool-settings presets, wherein the inferring includes inferring the medical-tool-settings preset usage values in the data set for different combinations of the users and the medical-tool-settings presets for which no medical-tool-settings preset usage currently exists responsively to the matrix factorization.

24. The method according to claim 19, further comprising:
inputting the data set into an artificial neural network (ANN); and
iteratively adjusting parameters of the ANN until an output of the ANN includes the input data set, the output including the inferred values.

25. The method according to claim 24, wherein the ANN includes an autoencoder.
